(19) United States
(12) Patent Application Publication
(10) Pub. No.: US 2008/0057003 A1
(43) Pub. Date: Mar. 6, 2008

(54) Powder inhaler formulations

(76) Inventors: Karoline Bechtold-Peters, Biberach (DE); Geoffrey Rowley, Ashbrooke (GB); Hanh Nguyen, Alnwich (GB)

Correspondence Address: MICHAEL P. MORRIS;BOEHRINGER INGELHEIM CORPORATION, 900 RIDGEBURY RD, P O BOX 368, RIDGEFIELD, CT, 06877-0368, US

(21) Appl. No.: 11/653,887

(22) Filed: Jan. 17, 2007

Related U.S. Application Data
(63) Continuation of application No. 11/093,491, filed on Mar. 30, 2005, now abandoned.
Application No. 11/093,491 is a continuation of application No. 10/085,972, filed on Feb. 28, 2002, now abandoned.

(30) Foreign Application Priority Data
Mar. 21, 2001 (GB) GB 01 07 106.7

(51) Int. Cl. A61K 9/14; A61K 31/38

(52) U.S. Cl. 424/46; 514/444

(57) ABSTRACT

The present invention relates to new methods for the surface modification of powders. Furthermore the present invention relates to new, improved pharmaceutical dosage forms obtainable by the new methods for surface modification of drugs according to the invention and to the use of these pharmaceutical dosage forms within dry powder inhalation devices (DPI).

The present invention relates to new methods for the surface modification of powders. Furthermore the present invention relates to new, improved pharmaceutical dosage forms obtainable by the new methods for surface modification of drugs according to the invention and to the use of these pharmaceutical dosage forms within dry powder inhalation devices (DPI).

### BACKGROUND OF THE INVENTION

Active substances for dry powder inhalation are often prepared by micronization or by spray drying to have an aerodynamic particle size of approximately 5 μm or less enabling lung deposition. Such powders present difficulties in manufacture and handling as well as in dispensing these powders during application due to particle agglomeration, cohesion and adhesion to manufacturing equipment, inhaler devices and container materials.

It is the object of the present invention to provide for new pharmaceutical dosage forms that are producible and applicable without displaying the drawbacks of conventional micronized or spray-dried powders for inhalation. In particular it is the object of the invention to provide for new pharmaceutical dosage forms being characterized by reduced electrostatic chargeability of the microfine active substances thereby improving powder flow properties during the manufacture of DPIs and improving powder dispensing and dispersion properties during application. Moreover, it is the object of the present invention to provide for a process of manufacture of these powders for inhalation.

### DESCRIPTION OF THE INVENTION

Surprisingly it has been found, that the aforementioned object of the invention is solved by an improved pharmaceutical dosage form for the use in a dry powder inhalation device (DPI) which comprises (a) at least one micronized or microfine solid active ingredient, which is soluble in water, (b) optionally a solid, pharmaceutically acceptable carrier excipient, which dilutes the active ingredient (a), (c) a fatty acid or fatty alcohol derivative or a poloxamer, characterized in that the fatty acid or fatty alcohol derivative or poloxamer (c) coats at least partially the surface of (a), |or of the agglomerate |formed |by (a) and (b)|.

Within the contents of this invention the micronized or microfine solid active ingredients are drugs for medical or diagnostic use. They are generally selected from those medicaments that are applicable via inhalation. Preferably they may be selected from the group consisting of anti-COPD-agents, anti-asthmatics, anti-migraine agents, anti-infective agents, anti-pain-agents, proteoglycans, therapeutic proteins, peptides and genes. Preferred active ingredients according to the invention are selected from the group consisting of beta-agonists such as Fenoterol, Formoterol and Salmeterol, anticholinergic drugs such as Ipratropium, Oxitropium, and Tiotropium, or combinations of beta-agonists and anticholinergics such as Tiotropium+Formoterol or Salmeterol, interferons such as interferon-alpha, interferon-beta, interferon-gamma or interferon-omega, cytokines such as interleukins and their antagonists or receptors, peptide hormones and analogues such as LHRH analogues, growth hormones and analogues, colony stimulating factors, erythropoietin, TNFs, vaccines, blood factors, enzymes, parathyroid hormone, calcitonin, insulin, antibodies such as antibodies to treat immune diseases, virus infections or lung cancer, alpha-1-antitrypsin, proteoglycans such as heparin or low molecular weight heparins, genes, anti-migraine drugs such as BIBN 4096, wherein Ipratropium, Tiotropium, Fenoterol, Salmeterol, Formoterol, or combinations of Tiotropium with Formoterol or Salmeterol, BIBN 4096, interferons, interleukin receptors and RSV-antibodies are the most preferred active ingredients.

Within the contents of this invention a reference to the aforementioned active ingredients is to be understood as reference to the active ingredients optionally in the form of their pharmaceutically acceptable acid addition salts, in the form of their solvates and hydrates.

The pharmaceutically acceptable acid addition salts are selected from the group consisting of hydrochloride, hydrobromide, sulfate, phosphate, methansulfonate, acetate, fumarate, lactate, citrate, tartrate and maleate. Preferred acid addition salts are selected form the group consisting of hydrochloride, hydrobromide, sulfate, phosphate and methansulfonate. More preferred acid addition salts are selected from the group consisting of hydrochloride, hydrobromide and methansulfonate.

If the active ingredient is selected from the group consisting of Ipratropium, Oxitroprium and Tiotropium reference to these ingredients is to be understood as reference to their salts selected from the group consisting of chloride, bromide, iodide, methansulfonate, para-toluenesulfonate or methylsulfate. In the aforemenetioned salts the active ingredients Ipratropium, Oxitroprium and Tiotropium represent kations. Preferred salts of Ipratropium, Oxitropium and Tiotropium are selected from the group consisting of chloride, bromide, iodide and methansulfonate, more preferred are methansulfonate and bromide, the latter one being most preferred.

The active ingredients used for the preparation of the pharmaceutical dosage forms according to the invention can optionally form solvates or hydrates. Accordingly, the term active ingredient not only relates to the salts and acid addition salts as specified hereinbefore, but embraces optionally existing solvates or hydrates thereof. In case of the preferred active ingredient Tiotropiumbromide the monohydrate thereof is of particular interest.

Within the contents of this invention acceptable carrier or, in the case of spray dried active ingredients, encapsulation excipients are selected from the group consisting of monosaccharides (e.g. glucose or arabinose), disaccharides (e.g. lactose, trehalose, sucrose, maltose), oligo- and polysaccharides (e.g. dextranes, hydroxyethyl cellulose), polyalcohols (e.g. sorbit, mannitol, xylit), salts (e.g. sodium chloride, calciumcarbonate), polyesters (e.g. polylactides and their copolymers), polyethers (e.g. PEG), sugar esters and ethers, polyvinyl derivatives (e.g. polyvinylalcohol) or mixtures thereof. Preferred acceptable carrier excipients are selected from mono- or disaccharides, especially lactose and glucose, optionally in the form of their hydrates. Of particular interest according to the invention are lactose-monohydrate and anhydrous glucose. Of particular interest as encapsulating agents are hydroxyethyl starch, trehalose, mannitol and lactose monohydrate or mixtures of mannitol and sucrose.

The average geometric particle size of the optionally added acceptable carrier excipients is in the range of 2-100 μm, preferably 4-60 μm, more preferably 6-40 μm, most preferably 8-35 μm. Of particular interest according to the invention are for example the following carrier excipients: Lactose monohydrate 200 mesh, optionally in mixture with micronized lactose, and glucose anhydrous 35 μm, optionally in mixture with micronized anhydrous glucose.

The average geometric particle size of the drug substance in line with this patent is 0.5-25 μm, preferably 1-20 μm, more preferably 1-15 μm. The average mass median aerodynamic diameter (MMAD) of the drug substance in this patent is targeted to be 0.5-15 μm, preferably 0.5-10 μm, more preferably 0.5-8 μm.

According to this invention, the term average geometric particle size is defined as the value in μm at which 50% of the particles as determined from the volume distribution of the particles by laser diffraction (dry suspension method) are smaller than or equal to this value. The MMAD in accordance with this patent is measured using appropriate devices such as cascade impactors or impingers as described and defined in the current pharmacopeias (e.g.: European Pharmacopoeia—Supplement 2001, pages 113-124 and 1657-1661).

According to the invention the the fatty acid or fatty alcohol derivatives or poloxamers are preferentially sorbitol derivatives, optionally containing polyethylene glycol ether groups, particularly they are selected from the group consisting of sorbitan mono-oleate, sorbitan trioleate, sorbitan monostearate, sorbitan tristearate, sorbitan monolaurate, sorbitan trilaurate, sorbitan monomyristate, sorbitan trimyristate, sorbitan monopalmitate, sorbitan tripalmitate, preferred PEG derivatives are PEG sorbitan monolaurate, PEG sorbitan monopalmitate, PEG sorbitan monostearate, PEG sorbitan tristearate, PEG sorbitan mono-oleate and PEG sorbitan trioleate. Preferred sorbitol derivatives are sorbitan mono-oleate, sorbitan trioleate sorbitan monostearate, sorbitan tristearate, PEG sorbitan monolaurate and PEG sorbitan mono-oleate, most preferred being sorbitan mono-oleate, sorbitan monostearate, sorbitan tristearate and PEG sorbitan mono-oleate.

Within the contents of the invention the term pharmaceutical dosage form is to be regarded as being equivalent to the term powder for inhalation.

The amounts of fatty acid or fatty alcohol derivative or poloxamer relative to the drug substance or—if carriers or encapsulating agents are present—relative to the drug substance plus excipient complex, i.e. the drug substance-excipient agglomerate or mixture or microcapsule, are in the range of 0.001-200% w/w, preferably 0.002-100% w/w, more preferably 0.01-50% w/w. Drug substance and surface modifying component together constitute 0.02-100% wlw, preferably 0.05-100% w/w, more preferably 0.1-100% w/w of the pharmaceutical dosage form.

The pharmaceutical dosage form according to the invention, is obtainable via processes of surface modification, involving the physical adsorption of a fatty acid or alcohol derivative or poloxamer (c) from solution or dispersion onto the surface of a drug (a), present as an insoluble particulate dispersion or by spray drying a solution or dispersion of the drug containing said fatty acid or alcohol derivative or poloxamer or by intensively physically mixing a powder containing the microfine drug with the fatty acid or alcohol derivative or poloxamer.

One process (process A) according to the invention comprises the steps of (i) preparation of a solution or dispersion of components (c)in a solvent, in which components (a) and optionally a carrier (b) are insoluble;

(ii) adsorption of components (c) to the surface of (a) and optionally (b) until equilibration;

(iii) separation of the dosage form by filtration and/or centrifugation, and

(iv) optionally drying of the resulting dosage form.

Another process (process B; spray drying process) according to the invention comprises the steps of:

(i) dissolving or dispersing components (a) and (c) in the solvent, optionally also adding encapsulating agents (d),

(ii) spray drying the solution or dispersion in a spray dryer under appropriate conditions resulting in microfine particles according to the particle size range described above

(iii) harvesting the spray dried particles in the cyclone or in the filter

(iv) optionally drying the particles to reach the wanted moisture content

(v) and finally optionally diluting the powder by addition of a carrier substance (b).

Another process (process C) according to the invention comprises the steps of:

(i) intensively mixing a powder containing the microfine drug substance (a), optionally also drug carrier (b), using standard mixing machines such as a Diosna mixer or a Lödige mixer,

(ii) either adding before start of the mixing process or, preferentially, during the mixing process components (c) to the powder and

(iii) running the mixing process for a while to enable that components (c) coat the surface of components (a) and optionally (b).

Another aspect of the invention relates to the processes of preparation of a pharmaceutical dosage form as described hereinbefore. Another aspect of the invention relates to a pharmaceutical dosage form obtainable via to the aforementioned process.

|In the first process according to the invention (process A), the active substances are water soluble and thus a non-aqueous solvent, preferably a water-immiscible organic solvent, was required for the adsorbate. Therefore, the solvent for step (i) in the first process (process A) is preferably a C₃-C₁₂ alkane or a C₃-C₁₂ cycloalkane, more preferably a C₅-C₈ alkane or a C₅-C₈ cycloalkane. The most preferred solvent is n-hexane or cyclohexane.|

In the second process according to the invention (process B) the solvent for step (i) in needs not to be a solvent in which for instance component (a) is insoluble. The solvent is preferably selected from water, aqueous buffer-solutions like for instance phosphate-buffer solutions, alcohols like for instance methanol, ethanol or isopropanol, C₃-C₁₂ alkanes, C₃-C₁₂ cycloalkanes or mixtures thereof. Preferred solvents for step (i) in process B are selected from water, aqueous buffer-solutions like phosphate-buffer solutions, alcohols and mixtures thereof, water and phosphate-buffer solutions being most preferred.

The concentration of the fatty acid or alcohol derivative or poloxamer in the solvent according to process A can vary from 20 mg/L to 10,000 mg/L, is preferably between 100 mg to 8,000 mg/L, more preferably between 200 mg and 5,000 mg/L, the most preferred concentration being 2000 mg/L.

In processes B and C the amount of fatty acid or fatty alcohol derivative or poloxamer added relative to the total solids is in the range of 0.001 to 50% w/w, preferably between 0.005 and 10% w/w, most preferred between 0.01 and 5% w/w.

In the processes according to the invention the drug substance is added in concentrations between 0.001% and 50%, preferably between 0.1% and 20%, the most preferred concentration is 2%, i.e. 4 g/200 ml.

From the aforementioned processes A, B, and C processes B and C are of particular interest, especially for processes in technical scale.

The pharmaceutical dosage forms display a variety of surprising and unexpected advantages and are therefore superior over conventional micronized and microfine powders for inhalation. By the surface modification of the active substances via adsorption of or coating by or intensive mixing with fatty acid derivatives the following effects proved to be of extraordinary significance:

(a) reduction of electrostatic charge acquisition by triboelectrification during pharmaceutical processing and during handling/drug administration,

(b) reduction of adhesion to contact surfaces,

(c) improvement of powder flow during pneumatic transport,

(d) improvement of drug content uniformity during mixing of actives with excipient carriers in DPI formulations and

(e) improvement of inhalation properties of powders.

The methods according to the invention generally provide for
the reduction of electrostatic charge acquisition by triboelectrification during pharmaceutical processing and during handling/drug administration, and
the reduction of adhesion to contact surfaces.

It is to be understood that these methods, even though being preferably applicable for the preparation and application of inhalation powders, are not limited to these powders.

Accordingly, a further aspect of the invention generally relates to a method for the reduction of electrostatic charge acquisition by triboelectrification during pharmaceutical processing and during handling/drug administration, characterized in that a surface modification involving the physical adsorption of a fatty acid or alcohol derivative or poloxamer from solution or dispersion onto the surface of a drug present as an insoluble particulate dispersion in the solution or the coating of the dissolved or dispersed drug by a fatty acid or alcohol derivative or poloxamer using spray drying or the intensve mixing of a drug containing powder with a fatty acid or alcohol derivative or poloxamer is conducted.

Another aspect of the invention generally relates to a method for the reduction of adhesion to contact surfaces, characterized in that a surface modification involving the physical adsorption of a fatty acid or alcohol derivative or poloxamer from solution or dispersion onto the surface of a drug present as an insoluble particulate dispersion in the solution or the coating of the dissolved or dispersed drug by a fatty acid or alcohol derivative or poloxamer using spray drying or the intensive mixing of a drug containing powder with a fatty acid or alcohol or poloxamerderivative is conducted.

Another aspect of the invention relates to a method for the improvement of powder flow during pneumatic transport, characterized in that a surface modification involving the physical adsorption of a fatty acid or alcohol derivative or poloxamer from solution or dispersion onto the surface of a drug present as an insoluble particulate dispersion in the solution or the coating of the dissolved or dispersed drug by a fatty acid or alcohol derivative or poloxamer using spray drying or the intensve mixing of a drug containing powder with a fatty acid or alcohol derivative or poloxamer is conducted.

Another aspect of the invention relates to a method for the improvement of drug content uniformity during mixing of actives with excipient carriers in DPI formulations, characterized in that a surface modification involving the physical adsorption of a fatty acid or alcohol derivative or poloxamer from solution or dispersion onto the surface of a drug present as an insoluble particulate dispersion in the solution or the coating of the dissolved or dispersed drug by a fatty acid or alcohol derivative or poloxamer using spray drying or the intensive mixing of a drug containing powder with a fatty acid or alcohol derivative or poloxamer is conducted.

Another aspect of the invention relates to a method for the improvement of inhalation properties of powders, characterized in that a surface modification involving the physical adsorption of a fatty acid or alcohol derivative or poloxamer from solution or dispersion onto the surface of a drug present as an insoluble particulate dispersion in the solution or the coating of the dissolved or dispersed drug by a fatty acid or alcohol derivative or poloxamer using spray drying or the intensve mixing of a drug containing powder with a fatty acid or alcohol derivative or poloxamer is conducted.

The advantages of the inhalation powders (pharmaceutical dosage forms) over conventional inhalation powders mentioned before are discussed and demonstrated in more detail below.

In the processing of micronized or microfine active substances for DPI, it is common to subject the powder to a sieving process in order to remove large agglomerates prior to mixing with the carrier particles used in the DPI formulation. Experimental evidence shows that sieved untreated samples have greater electrostatic charge acquisition by a process of triboelectrification against a contact surface of stainless steel in a cyclone separator. The experimental method for electrostatic charge determinations that was applied is outlined in more detail below.

Comparison of sieved samples of unmodified active and active modified by the adsorption process shows considerable differences in acquired charge. The method applied for the preparation of sieved powder samples is outlined in detail below.

### BRIEF DESCRIPTION OF THE DRAWINGS

FIG. 1: Mean specific charge of micronized Fenoterol generated during triboelectrification in a stainless steel cyclone with or without sieving and with and without organic solvent/antistatic agent treatment;

FIG. 2: Mass of micronized Fenoterol (1 g samples) transported to the Faraday well during triboelectrification in a stainless steel cyclone with or without sieving and with and without organic solvent/antistatic agent treatment;

FIG. 3: Mean specific charge of micronized Tiotropium generated during triboelectrification in a stainless steel cyclone with or without sieving and with and without organic solvent/antistatic agent treatment;

FIG. 4: Mass of micronized Tiotropium (1 g samples) transported to the Faraday well during triboelectrification in a stainless steel cyclone with or without sieving and with and without organic solvent/antistatic agent treatment;

FIG. 5: Mean specific charge after mixing in Turbula mixer (Fenoterol and Ipratropium);

FIG. 6: Mean specific charge after mixing in Turbula mixer (Tiotropium and Oxitropium);

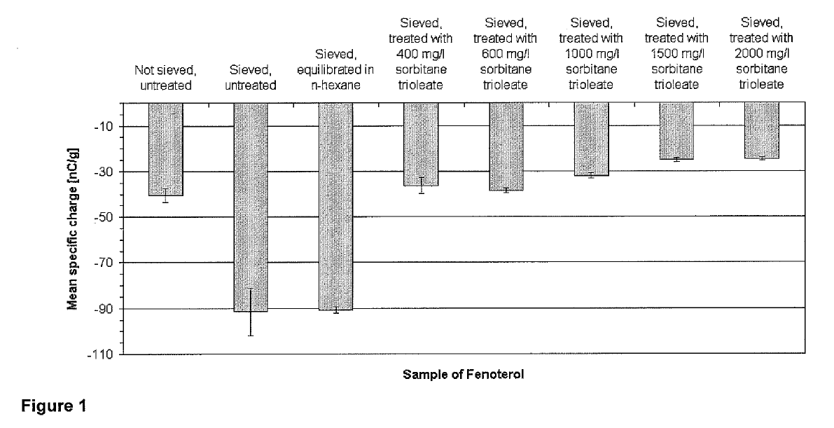

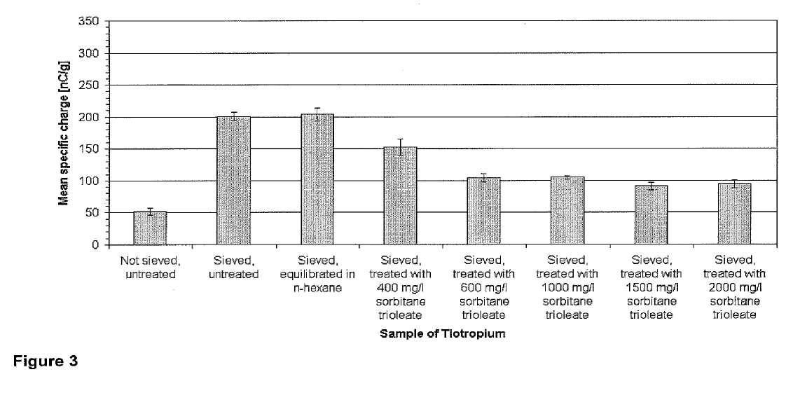

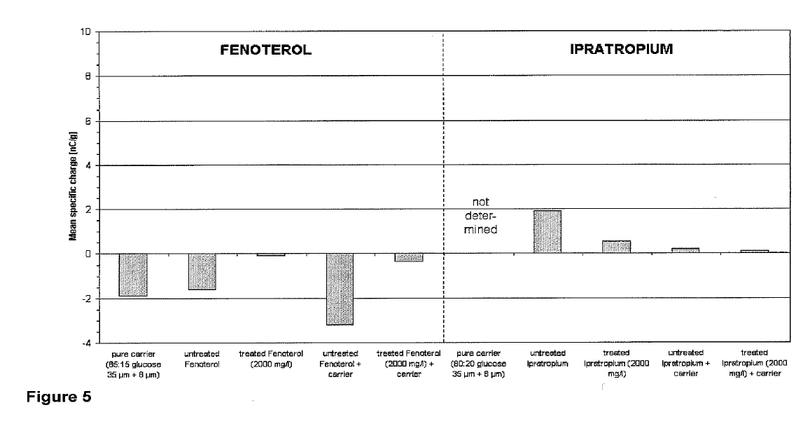

FIG. 1 provides specific charge values of −40 and −92 nC g⁻¹ for unsieved and sieved fenoterol respectively and the charge values in FIG. 3 for unsieved and sieved tioptropium were +52 and +201 nC g⁻¹ respectively. FIGS. 1 and 3 show that treatment of the active substances with sorbitan trioleate reduces charge acquistion of sieved samples when using the same process of triboelectrification. An example from these data in FIGS. 3 and 5 shows the mean charge values for the drugs fenoterol and tiotropium when treated at a concentration of 600 mg l⁻¹ of sorbitan trioleate in hexane. Sieved samples of the treated fenoterol and tiotropium had mean charge values of −38.4 and +104 nC g⁻¹ respectively, after triboelectrification in the cyclone apparatus. These data show that charge acquisition for sieved samples can be reduced by surface modification.

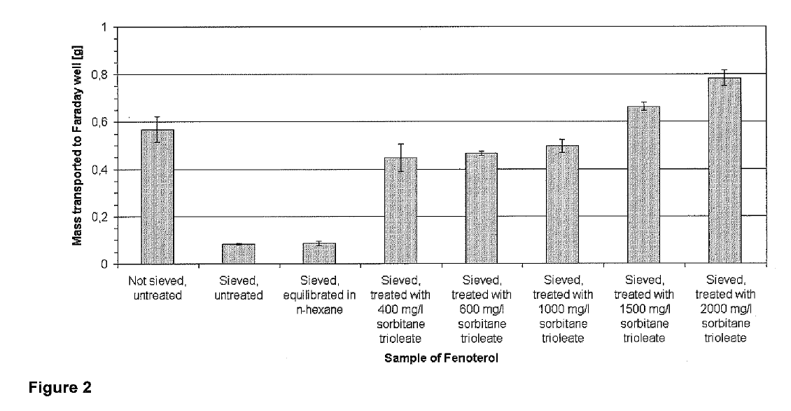

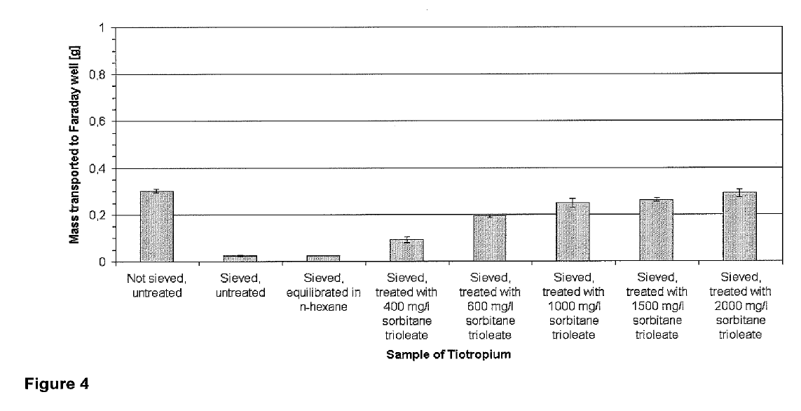

Experimental results show that sieving also adversely affects bulk powder properties of the active substances, including adhesion to contact surfaces and pneumatic flow. FIGS. 2 and 4 provide mass transfer values of powder through the cyclone apparatus by pneumatic conveyance during triboelectrification, experiments. Ideally, 100% w/w of the original sample (1 g) should pass through the apparatus and this would indicate good flow and non-adhesion.

FIGS. 2 and 4 provide values of mass transfer of 0.083 and 0.025 g (8.3 and 2.5% w/w) for sieved, untreated fenoterol and tiotropium respectively. Treatment of the actives by surface modification with sorbitan trioleate increased the mass transfer values to an extent that was dependent upon treatment concentration. FIG. 2 shows increases in mass transfer to between 0.45-0.78 g (45-78% w/w) for fenoterol and in FIG. 4 the values increase to between 0.092-0.29 g (9.2-29% w/w) for tiotropium.

Visual inspection of the steel contact surface showed that powder adhesion was considerably less for surface modified actives. In addition, the adhered treated samples were very easily removed, whereas untreated actives were firmly adhered and very difficult to remove.

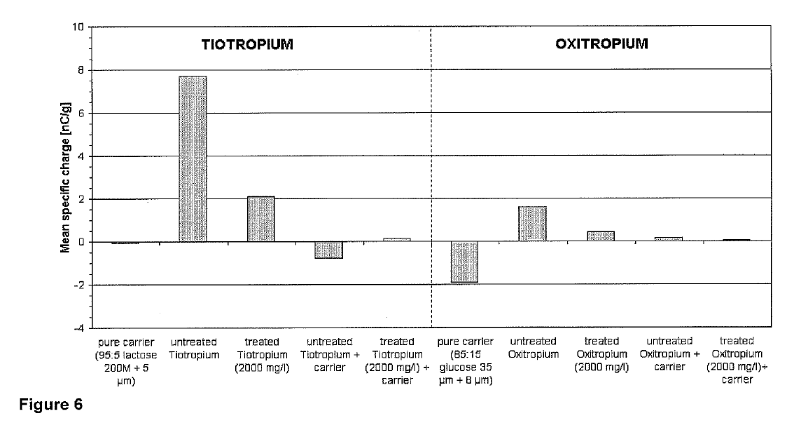

Triboelectrification of powders occurs during mixing processes. FIGS. 5 and 6 show values for charge acquisition for powder samples of, (a) carrier excipients, (b) untreated and treated actives and (c) DPI formulations of untreated and treated actives. The results in these figures show that the treatment by adsorption of sorbitan trioleate reduces charge acquisition of both the unformulated and formulated actives during mixing in a steel mixing vessel of a turbula mixer( for method see experimental part III). Untreated fenoterol in a DPI formulation with glucose as carrier had a mean specific charge of −3.2 nC g⁻¹, whereas the formulation containing treated drug had a value of −0.35 nC g⁻¹ (FIG. 5). Tiotropium (untreated) in DPI formulation with lactose as carrier had a mean charge value of −0.78 nC g⁻¹ and the formulation containing treated drug had a value of 0.15 nC g⁻¹ (FIG. 6).

DPI formulations containing untreated and treated actives were prepared by mixing in a steel vessel of a turbula mixer and 20 random samples from each mix were analysed for the active component. The methodology applied is outlined in detail below. The mean drug content and coefficient of variation (cv) values in table 1 show that the treatment of tiotropium with sorbitan trioleate improves the mixing quality and hence the drug content uniformity.

TABLE 1
Mean drug content and coefficient of variation values for DPI
formulations prepared in a turbula mixer:

| | Mean drug | |
|---|---|---|
| DPI formulation | content (mg) | cv (%) |
| Untreated tiotropium | 0.24 | 45.8 |
| Treated tiotropium (sorbitan | 0.22 | 4.5 |
| trioleate at 2000 mg/L | | |
| concentration) | | |
| Untreated fenoterol | 2.1 | 30.9 |
| Treated fenoterol (sorbitan | 2.0 | 4.0 |
| trioleate at 2000 mg/L | | |
| concentration) | | |

The effect of different sorbitan derivatives on charge and mass transfer is summarized in table 2 for tiotropium. In all cases, the charge value acquired by triboelectrification in the cyclone apparatus is lower than for untreated tiotropium. The mass transfer values indicate that sorbitan mono-oleate is the most effective derivate for charge reduction and there is little difference in effectiveness between the stearate derivatives.

TABLE 2
Mean charge nC g⁻¹(cv %), mass transfer (% w/w) (cv %) for sieved
samples of untreated tiotropium and tiotropium treated with sorbitan
derivatives at 600 mg l⁻¹

| | Mean charge | Mean mass |
|---|---|---|
| Sorbitan derivative | nC g⁻¹ | transfer % |
| Mono-oleate | +39.6 (4.6) | 53 (3.8) |
| Trioleate | +104.5 (5.9) | 19.4 (3.1) |
| Monostearate | +75.1 (1.1) | 33 (9.1) |
| Tristearate | +70.1 (3.1) | 17 (11.8) |
| Untreated tiotropium | +201 (3.2) | 2.5 (8.0) |

### EXPERIMENTAL PART

I. ELECTROSTATIC CHARGE DETERMINATIONS

Triboelectrification in a Cyclone Separator

Electrostatic charge of powder samples was investigated using a cyclone apparatus linked to a Faraday well and force compensation load cell to measure charge and mass simultaneously. 1 g samples of powder were transported through the apparatus using dry compressed air (rh<10%) at 8 m s⁻¹ for triboelectrification against a stainless steel surface.

The charge Q (nC) and mass M (g) values were used to calculate the specific charge Q/M (nC g⁻¹) at the completion of each experimental run. The results are mean values with coefficient of variation values for 5 replicates. The mass of material entering the Faraday well was used to quantify the mass transport through the apparatus and this was used to assess the flow and adhesion characteristics of the powder. In addition, the amount of material adhered to the cyclone wall was estimated visually and rated on a scale from 0 (no adhesion) to 3 (extensive adhesion).

Triboelectrification in a Turbula Mixer

The electrostatic charge of the drug/carrier powder mixes (5 g) was undertaken after mixing in a stainless steel cylindrical vessel, agitated at 100 rpm for 10 minutes on a Turbula mixer under ambient conditions, by pouring the sample into a Faraday well. The mass of powder entering the Faraday well was recorded to determine the specific charge. In addition, the difference between the mass of powder in the mixing vessel and that in the Faraday well was used to quantify the amount of adhesion to the mixer vessel wall. The mean specific charge, and coefficient of variation values for 3 replicates are reported.

### II. PREPARATION OF SIEVED POWDER SAMPLES

Approximately 10 g of drug powder samples were placed in a 60M (250 μm) sieve and agitated using a sieve shaker (Glen Creston, 47-300) with an oscillation amplitude regulator at setting 20 for 20 minutes. Sieved powder samples were stored in glass jars and then kept in a desiccator for a week prior to charge investigations in the cyclone.

### III: EFFECTS OF MIXING

Effect on Charging

The untreated and treated active substances were mixed with carrier excipient in a ratio selected from the range of drug/carrier compositions used in dry powder inhaler formulations. A carrier blend of coarse and micronized carrier was prepared in a turbula mixer for 10 minutes at 100 rpm. The active substance (treated or untreated drug) was added and mixed for further 10 minutes prior to charging measurements.

Effect of Treatment on Drug Content and Uniformity

The untreated and treated drugs were mixed with carrier excipient as follows.
5.2036 g lactose 200M:
0.2739 g micronized lactose:
0.0225 g untreated or treated Tiotropium
4.4880 g glucose 35 μm:
0.7920 g glucose 15 μm:
0.2200 g untreated or treated Fenoterol (total mixing time reduced to 10 minutes, comprising 5 for carrier blend and 5 for carrier/active blend.)

20 samples, approximately 50mg, were taken at random from each mixed formulation, accurately weighed and dissolved in 20 ml distilled water. Drug concentration in each sample was determined spectrophotometrically at λₘₐₓ 237 nm and 276 nm for tiotropium and fenoterol respectively. A modified BP content uniformity was applied (20 samples were examined). The mean drug content and coefficient of variation were calculated.

### IV: PREPARATION OF PHARMACEUTICAL DOSAGE FORMS

Starting Materials:

The starting materials are unless otherwise specified commercially available or obtainable via convenional methods known in the art.

Tiotropiumbromide Monohydrate:

15.0 kg Tiotropiumbromide are introduced into 25.7 kg water. The mixture is heated to 80-90° C. and stirred at that temperature until a clear solution is obtained. Charcoal (0.8 kg) is introduced into 4.4 kg water and the mixture thus obtained is added to the aforementioned solution of tiotropiumbromide. The obtained reaction mixture is stirred for at least 15 min at 80-90° C. and is, subsequently, hot-filtered into another reaction apparatus being preheated to about 70° C. The filter is washed with 8.6 kg of water. The mixture thus obtained is cooled to about 20-25° C. (3-5° C. per 20 minutes). The crystallization is completed by stirring at the aforementioned temperature for at least 1 hour. The crystalline product is isolated and washed with 9 L of cold water (10-15° C.) and cold acetone (10-15° C.). The crystals are dried for 2 hours at about 25° C. under nitrogen. Yield: 13.4 kg tiotropiumbromide monohydrate (86%).

The crystalline tiotropiumbromide monohydrate thus obtained is micronized according to conventional methods known in the art.

Preparation of Formulation Via Physical Adsorption:

4 g of drug were equilibrated with adsorbate in hexane in a concentration range from 200 to 2×10³ mg l⁻¹ in an incubator agitated at 220 rpm for 3 hours at 25±0.5° C. The treated drug was filtered using vacuum and dried in a fume cupboard to constant weight at room temperature. Dried treated drugs were lightly milled using a mortar and a pestle.

Preparation of Formulation Via Spray Drying:

Up to 20 g solids including the drug substance, the embedding agent and 0.001 to 2% (w/100 ml) of the fatty acid or alcohol derivative or poloxamer were dissolved or dispersed in water or aqueous buffer solution, e.g. 20 mM phosphate buffer, in an alcohol, a ketone, a hydrocarbon or halogenated hydrocarbon, or in a mixture thereof. The mixture was spray dried using an appropriate spray dryer such as a Büchi Mini SprayDrier, a Niro SDMicro or a Niro Mobile Minor, and harvested from the cyclon or the filter or both. The resulting powder may be vacuum dried at 40° C. to reduce residual moisture.

V. EXAMPLES FOR FORUMULATION OF PHARMACEUTICAL DOSAGE FORMS PREPARED IN LINE WITH THIS PATENT

### Example 1

4 g Fenoterol hydrobromide are dispersed in an incubator in 200 ml of n-hexane containing 2000 mg/L sorbitan trioleate and agitated at 220 rpm for 3 hours at 25±0.5° C. The treated drug is filtered using vacuum and dried in a fume cupboard to constant weight at room temperature, followed by lightly milling using a mortar and a pestle and sieving through a 250 μm sieve. Electrostatic charge after one week storage in a dessicator at room temperature: −24.7 nC/g specific charge and 78.3% transported mass.

Composition of Formulation:

0.2200 g Fenoterol hydrobromide, treated with sorbitan trioleate (see hereto above);

4.4880 g Glucose 35 μm;

0.7920 g micronized Glucose;

The components are carefully mixed and filled into capsules or blisters for use in commercial inhaler devices

### Example 2

4 g Tiotropiumbromide monohydrate are dispersed in an incubator in 200 ml of n-hexane containing 3000 mg/L sorbitan trioleate and agitated at 220 rpm for 3 hours at 25±0.5° C. The treated drug is filtered using vacuum and dried in a fume cupboard to constant weight at room temperature, followed by lightly milling using a mortar and a pestle and sieving through a 250 μm sieve. Electrostatic charge after one week storage in a dessicator at room temperature: −96.4 nC/g specific charge and 13.5% transported mass.

Composition of Formulation:

0.0225 g Tiotropiumbromide monohydrate, treated with sorbitan trioleate (see hereto above);

5.2036 g Lactose 200 M;

0.2739 g micronized lactose;

The components are carefully mixed and filled into capsules or blisters for use in commercial inhaler devices.

### Example 3

4 g Tiotropiumbromide monohydrate are dispersed in an incubator in 200 ml of n-hexane containing 2000 mg/L sorbitan monostearate and agitated at 220 rpm for 3 hours at 25±0.5° C. The treated drug is filtered using vacuum and dried in a fume cupboard to constant weight at room temperature, followed by lightly milling using a mortar and a pestle and sieving through a 250 μm sieve. Electrostatic charge after one week storage in a dessicator at room temperature: −31.4 nC/g specific charge and 63.7% transported mass.

Composition of Formulation:

0.0225 g Tiotropiumbromide monohydrate, treated with sorbitan monostearate (see hereto above);

5.2036 g Lactose 200 M;

0.2739 g micronized lactose;

The components are carefully mixed and filled into capsules or blisters for use in commercial inhaler devices.

### Example 4

4 g Tiotropiumbromide monohydrate are dispersed in an incubator in 200 ml of n-hexane containing 2000 mg/L sorbitan mono-oleate and agitated at 220 rpm for 3 hours at 25±0.5° C. The treated drug is filtered using vacuum and dried in a fume cupboard to constant weight at room temperature, followed by lightly milling using a mortar and a pestle and sieving through a 250 μm sieve. Electrostatic charge after one week storage in a dessicator at room temperature: −31.4 nC/g specific charge and 60.0% transported mass.

Composition of Formulation:

0.0225 g Tiotropiumbromide monohydrate, treated with sorbitan mono-oleate (see hereto above);

5.2036 g Lactose 200 M;

0.2739 g micronized lactose;

The components are carefully mixed and filled into capsules or blisters for use in commercial inhaler devices.

### Example 5

4 g Oxitropiumbromide are dispersed in an incubator in 200 ml of n-hexane containing 2000 mg/L sorbitan trioleate and agitated at 220 rpm for 3 hours at 25±0.5° C. The treated drug is filtered using vacuum and dried in a fume cupboard to constant weight at room temperature, followed by lightly milling using a mortar and a pestle and sieving through a 250 μm sieve. Electrostatic charge after one week storage in a dessicator at room temperature: 78.7 nC/g specific charge and 33.1% transported mass.

Composition of Formulation:

0.11 g Oxitropiumbromide, treated with sorbitan trioleate (see hereto above);

4.5815 g Glucose 35 μm;

0.8085 g micronized glucose;

The components are carefully mixed and filled into capsules or blisters for use in commercial inhaler devices.

### Example 6

4 g Ipratropiumbromide are dispersed in an incubator in 200 ml of n-hexane containing 2000 mg/L sorbitan trioleate and agitated at 220 rpm for 3 hours at 25±0.5° C. The treated drug is filtered using vacuum and dried in a fume cupboard to constant weight at room temperature, followed by lightly milling using a mortar and a pestle and sieving through a 250 μm sieve. Electrostatic charge after one week storage in a dessicator at room temperature: 78.2 nC/g specific charge and 34.2% transported mass.

Composition of Formulation:

0.2296 g Ipratropiumbromide, treated with sorbitan trioleate (see hereto above);

4.2163 g Glucose 35 μm;

1.0541 g micronized glucose;

The components are carefully mixed and filled into capsules or blisters for use in commercial inhaler devices

### Example 7

10 g of trehalose is dissolved in 50 ml of 20 mM phosphate buffer pH 5.5 containing 0.1% Tween 80 (PEG sorbitan mono-oleate). 50 ml of a solution of 55 mg of Interferon-omega in 20 mM phosphate buffer pH 5.5 is slowly added under gentle stirring. The solution is spray dried at 90° C. inlet temperature and 60° C. outlet temperature. The almost free flowing powder is easily harvested from the cyclon and dried under vacuum for 6 hours at 40° C. The powder is filled into capsules, but may be diluted by carrier 1:10 prior to filling into the capsules.

### Example 8

10 g of hydroxyethyl starch is dissolved in 100 ml of 20 mM phosphate buffer pH 5.5 containing 0.5% Tween 80 (PEG sorbitan mono-oleate). 100 ml of a solution of 55 mg of Interferon-omega in 20 mM phosphate buffer pH 5.5 is slowly added under gentle stirring. The solution is spray dried at 90° C. inlet temperature and 60° C. outlet temperature. The powder is harvested from the cyclon and dried under vacuum for 6 hours at 40° C. The powder is filled into capsules, but may be diluted by carrier 1:10 prior to filling into the capsules.

1. A pharmaceutical dosage form for use in a dry powder inhalation device which comprises, in dry powder form:
(a) at least one micronized or spray dried solid active ingredient, which active ingredient is soluble in water; and
(b) a coating material selected from the group consisting of a fatty acid sorbitan ester or a PEG ether thereof and a poloxamer,
wherein the coating material coats at least partially the surface of the active ingredient, wherein the pharmaceutical dosage form has an average mass median aerodynamic diameter (MMAD) of 0.5-15 μm, and wherein the active ingredient is a salt selected from ipratropium, oxitropium or tiotropium salts.

2. The pharmaceutical dosage form as recited in claim 1 wherein the active ingredient has been encapsulated and the coating material partially coats the so-encapsulated active ingredient.

3. The pharmaceutical dosage form as recited in claim 1 further comprising a solid, pharmaceutically acceptable carrier excipient and the coating material coats at least partially the surface of the agglomerate or the mixture formed by the active ingredient and the carrier excipient.

4. The pharmaceutical dosage form as recited in claim 1 wherein the active ingredient has a mean mass aerodynamic diameter of about 0.5 to about 8 μm.

5. (canceled)

6. The pharmaceutical dosage form as recited in claim 1, wherein the coating material is selected from the group consisting of sorbitan mono-oleate, sorbitan trioleate, sorbitan monostearate, sorbitan tristearate, sorbitan monolaurate, sorbitan trilaurate, sorbitan monomyristate, sorbitan trimyristate, sorbitan monopalmitate, sorbitan tripalmitate, PEG sorbitan monolaurate, PEG sorbitan monopalmitate, PEG sorbitan monostearate, PEG sorbitan tristearate, PEG sorbitan mono-oleate and PEG sorbitan trioleate.

7. The pharmaceutical dosage form according to claim 1, wherein the active ingredient is a salt of ipatropium.

8. The pharmaceutical dosage form according to claim 1, wherein the active ingredient is a salt of oxitropium.

9. The pharmaceutical dosage form according to claim 1, wherein the active ingredient is a salt of tiotropium.

10. The pharmaceutical dosage form according to claim 1, wherein the solid active ingredient is micronized.
